(19) United States
(12) Patent Application Publication
(10) Pub. No.: US 2011/0040266 A1
(43) Pub. Date: Feb. 17, 2011

(54) ANTI-INSOMNIA COMPOSITIONS AND METHODS

(76) Inventors: Frank E. Blondino, Easton, PA (US); David Bergstrom, Flemington, NJ (US); Foyeke Opawale, Flemington, NJ (US)

Correspondence Address: DICKSTEIN SHAPIRO LLP, 1825 EYE STREET NW, Washington, DC, 20006-5403, US

(21) Appl. No.: 12/912,261

(22) Filed: Oct. 26, 2010

Related U.S. Application Data
(62) Division of application No. 12/119,030, filed on May 12, 2008.
(60) Provisional application No. 60/917,243, filed on May 10, 2007.

(51) Int. Cl. A61M 21/02; A61M 35/00

(52) U.S. Cl. 604/310

(57) ABSTRACT

Compositions of zolpidem, and methods for their manufacture and use for treating insomnia. The compositions are formulated as oral sprays for transmucosal absorption of zolpidem. The methods of treatment in some cases involve night-time dosing administration to achieve therapeutic zolpidem blood levels within 20 minutes or less, tapering off to less than 20 ng/ml within less than five hours, in some cases less than four hours, post dosing.

The present invention claims priority to U.S. Provisional Application Ser. No. 60/917,243 filed May 10, 2007. The disclosure of this provisional and of U.S. Patent Publication No. 2006/0216240 A1 are hereby incorporated by reference herein in their entireties.

### FIELD OF THE INVENTION

The present invention relates to compositions of zolpidem, and methods for their manufacture and use for treating insomnia.

### BACKGROUND OF THE INVENTION

Zolpidem, N,N,6-trimethyl-2-p-tolyl-imidazo[1,2-a]pyridine-3-acetamide, is a non-benzodiazepine sedative-hypnotic. Zolpidem is available as an oral tablet to treat insomnia at a dose of between 5 and 12.5 mg. Typically, zolpidem is administered as the tartrate salt, i.e., N,N,6-trimethyl-2-p-tolyl-imidazo[1,2-a]pyridine-3-acetamide L-(+)-tartrate (2:1). Tolerance and physical dependence is only rarely observed with zolpidem. (See e.g., Goodman and Gilman's The Pharmacological Basis of Therapeutics, 9^{th} ed., pp. 471-472).

The side effects of zolpidem, however, can include daytime drowsiness. As reported by Hindmarch et al. (Residual effects of zaleplon and zolpidem following middle of the night administration five hours to one hour before awakening, Hum. Psychopharcol., 2001 Mar., 16(2): 159-167) the entirety of which is hereby incorporated herein by reference, the night-time dosing administration of zolpidem in tablet form results in residual drowsiness and sleepiness when administered from 5 hours to 1 hour before waking. Accordingly, the instructions for use of Ambien®, a commercial zolpidem product, state: “Do not take Ambien or any other sleep medicine unless you are able to get a full night's sleep before you must be active again.” (Physician's Desk Reference, Jan. 3, 1997 at 2932).

### BRIEF DESCRIPTION OF THE DRAWINGS

FIG. 1 is a graphic representation of the means and standard errors of zolpidem concentration levels during the first 30 minutes post-dosing for Study 1, described below.

FIG. 2 is a graphic representation of the number of test subjects that achieve levels greater than approximately 4.7 ng/mL of zolpidem at certain time intervals post-dosing for Study 1.

FIG. 3 is a graphic representation of the drowsiness/sleepiness score 15 minutes post-dosing for Study 1.

FIG. 4 is a graphic representation of zolpidem concentration levels during the first 60 minutes post-dosing for Study 2.

FIG. 5 is a graphic representation of zolpidem concentration levels during the first 30 minutes post-dosing for Study 2.

FIG. 6 is a graphic representation of the AUC up to 30 minutes post-dosing for Study 2.

FIG. 7 is a graphic representation of plasma profile of zolpidem following administration of 5 mg of zolpidem by oral spray (“LS”) under fasting conditions.

FIG. 8 is another graphic representation of plasma profile of zolpidem following administration of 5 mg of zolpidem LS under fasting conditions.

FIG. 9 is another graphic representation of plasma profile of zolpidem following administration of 5 mg of zolpidem LS under fasting conditions.

FIG. 10 is a graphic representation of plasma concentration of zolpidem following administration of 5 mg of zolpidem LS under fasting conditions.

FIG. 11 is a graphic representation of simulated plasma concentration of zolpidem following administration of 2.5 mg and 5.0 mg zolpidem LS at 4 hour intervals.

FIG. 12 is another graphic representation of simulated plasma concentration of zolpidem following administration of 2.5 mg and 5.0 mg zolpidem LS at 4 hour intervals.

### SUMMARY OF THE INVENTION

The invention relates to compositions and methods for inducing sleep by administering a dose of an oral spray composition to a patient suffering from insomnia. The composition contains a sedative or a pharmaceutically acceptable salt thereof and a pharmaceutically acceptable solvent. The spray is administered, in some cases, within less than about five hours before the patient needs to arise from sleep and resume wakeful activities.

In some cases, the dose comprises about 2.0 to about 3.0 mg of zolpidem or a pharmaceutically acceptable salt thereof, and the dose has a volume in the range from about 50 to about 400 mcL. In other cases, the dose is about 2.5 mg and the volume of a unit dose spray is about 50 mcL.

The solvent may comprise a polar solvent or a non-polar solvent. The composition may optionally comprise a taste mask or flavoring agent, a propellant, and other excipients.

In one embodiment, the method includes administering to a patient suffering from insomnia a volume of about 50 to about 400 mcL of a composition by oral spray for transmucosal absorption to the patient's systemic circulatory system. The composition contains a dose of zolpidem or a pharmaceutically acceptable salt thereof and a pharmaceutically acceptable solvent adapted for transmucosal absorption of zolpidem through the oral mucosa to the patient's systemic circulatory system. The dose may, in some cases, be between about 0.5 mg and about 5.0 mg of zolpidem or a pharmaceutically acceptable salt thereof, and be administered within less than about four or about five hours before the patient needs to arise from sleep. In some cases, the administration by oral spray results in therapeutic blood levels of zolpidem within 12, 13, 22, or 23 minutes and tapers off to less than 20 ng/ml less than five hours post dosing.

In some cases the dose comprises between about 0.5 to 2.5 mg of zolpidem or a pharmaceutically acceptable salt thereof, and is administered by oral spray within less than about four hours before the patient needs to resume wakeful activities.

In some cases, the composition comprises about 1.0 to about 10.0 weight percent zolpidem or a pharmaceutically acceptable salt thereof; about 40 to about 60 percent water; and about 20 to 50 percent solvent. In other cases, the composition comprises about 3.0 to about 7.0 percent zolpidem or a pharmaceutically acceptable salt thereof; about 45 to about 50 percent water; about 30 to 40 percent solvent.

In some cases, a therapeutic blood level of zolpidem is achieved within less than about 20 minutes and tapers off to less than 20 ng/ml within less than four hours post dosing.

In some cases, the composition comprises zolpidem or a pharmaceutically acceptable salt thereof, and a pharmaceutically acceptable solvent, wherein the composition is contained in a unit dose spray pump container. A single actuation of such container delivers a unit dose volume about 50 to about 400 mcL of the composition, containing a dose of about 0.5 to 5 mg zolpidem or a pharmaceutically acceptable salt thereof. In other cases, the unit dose volume is about 50 to about 200 mcL, and/or the dose of zolpidem is about 2 to 3 mg.

### DETAILED DESCRIPTION OF PREFERRED EMBODIMENTS

Embodiments of the present invention provide a spray composition which provides a biologically active sleep-inducing compound for rapid absorption through the oral mucosa of a human patient, resulting in fast onset of effect. Embodiments of the invention relate to night-time dosing to treat insomnia in a patient by spraying the oral mucosa of the patient with a therapeutically effective amount of an oral spray comprising zolpidem or a pharmaceutically acceptable salt thereof. The invention also provides methods of treating insomnia using such compositions during night-time dosing or other times when a patient cannot obtain a full night's sleep prior to being active again.

By “night-time dosing” or “middle of the night dosing” herein we mean providing a pharmaceutically effective dose for treating insomnia when a patient cannot obtain a full night's sleep after such dose is administered and before the patient must be active again. Night-time dosing can include dosing at, for example, 2:00 am, 3:00 am, midnight to 4:00 am, or etc., and also extends to providing a dosage during the day when the patient, due to his or her occupation, travel, or other activities, needs to be active during the night and typically obtains sleep during daylight hours. Therefore, night-time dosing or middle of the night dosing as used herein includes administering a dose at any time when the patient must be active again within about four hours, or less than about five to six hours, of such dose.

The doses of zolpidem according to some embodiments can be about 0.5 mg to about 10.0 mg (e.g., 0.5 mg, 1.0 mg, 2.0 mg, 2.5 mg, 3.0 mg, 4.0 mg, 5.0 mg, or 10.0 mg) zolpidem or pharmaceutically acceptable salt, such as, for example, 0.5 to 2.5 mg or more zolpidem tartrate.

When zolpidem or a pharmaceutically acceptable salt thereof is the active compound, the spray may contain from about 0.01 to 20 weight/weight (w/w) percent zolpidem, 0.1 to 15 w/w percent zolpidem, or 0.5 to 5 w/w percent zolpidem. The term “pharmaceutically acceptable salts” refers to salts prepared from pharmaceutically acceptable non-toxic acids or bases including organic and inorganic acids or bases.

The oral spray compositions may further comprise a pharmacologically acceptable polar or non-polar solvent, or mixture thereof. The solvent may comprise a polar solvent, for example, between about 10-99 weight % of total composition. Optionally, the composition can further comprise a propellant, for example, between about 2-90 weight % of total composition. Also optionally, a taste mask and/or flavoring agents may be included, for example between about 0.01-10 weight % of total composition. Preservative(s) may also be optionally included, for example between about 0.001-1 weight % of total composition.

According to one embodiment, an oral spray composition for transmucosal administration of zolpidem comprises in weight % of total composition: 0.05-10% zolpidem or a pharmaceutically acceptable salt thereof; 88-99.05% of a polar or non-polar solvent or mixture thereof; 0-1% taste mask and/or flavoring agents; and 0-1% preservative.

A further embodiment provides an aerosol valved container containing a propellant, a solvent composition, and the active agent. As the propellant evaporates after activation of the aerosol valve, a mist of droplets is formed which contains solvent and active compound.

The formulations may contain an optional propellant for delivery as an aerosol spray, or can be propellant-free and delivered by a metered valve spray pump. Suitable propellants include, but are not limited to, hydrocarbons (butane, propane, etc.), chlorofluorocarbons (CFC-11, CFC-12, etc.), hydrofluorocarbons (HFA-134a, HFA-227ea, etc.), and ethers (dimethylether, diethylether, etc.). The propellant may be substantially non-aqueous. The propellant produces a pressure in the aerosol container such that under normal usage it will produce sufficient pressure to expel the solvent from the container when the valve is activated but not excessive pressure such as to damage the container or valve seals.

The non-polar solvent is in some cases a non-polar hydrocarbon, such as a C₇₋₁₈ hydrocarbon of a linear or branched configuration, fatty acid esters, and triglycerides such as MIGLYOL®. Suitable non-polar solvents, may, for example, include (C₂-C₂₄) fatty acid (C₂-C₆) esters, C₇-C₁₈ hydrocarbon, C₂-C₆ alkanoyl esters, and the triglycerides of the corresponding acids. The solvent should preferably dissolve the active compound and be miscible with the propellant, i.e., solvent and propellant should preferably form a single phase at a temperature of 0-40° C. at a pressure range of between 1-10 atm.

Solvents for the polar sprays include, for example, low molecular weight polyethyleneglycols (PEG) of 300-1000 Mw (preferably 400-600); low molecular weight (C₂-C₈) mono and polyols; and alcohols of C₇-C₁₈ linear or branch chain hydrocarbons; and/or glycerin and water. Many other suitable polar and non-polar solvents may be utilized, such as acidified water and/or aqueous buffers.

The polar and non-polar aerosol spray compositions of the invention may be administered from a sealed, pressurized container. The contents of the container are released by activation of a metered valve, which does not allow entry of atmospheric gasses with each activation.

A further embodiment provides a pump spray container containing a composition of the pump spray formulation, and a metered valve suitable for releasing from the container a predetermined amount of said composition. The compositions stored in the container may be at or below atmospheric pressure.

Yet another embodiment provides a method of treating insomnia with night-time dosing of zolpidem administered to the oral mucosa (i.e., buccal, lingual, and/or sublingual, etc. mucosa) by spray. Preferred embodiments administer a spray volume of about 25-400 mcL, about 50-200 mcL, or about 100 mcL to the oral mucosa. In another embodiment the spray volume is about 50 mcL. The volume of spray may contain a dose of zolpidem in the range from, for example, about 0.5 mg to about 10.0 mg. The dose may be administered about 2, 3, less than about 5 or 6 hours prior to the patient being active again.

The active compound may include zolpidem base and its derivatives, such as zolpidem tartrate, and/or other pharmaceutical acceptable salts or other forms thereof. In a preferred embodiment, the active compound is zolpidem tartrate.

The active compounds may be in an ionized, salt form or as the free base of the pharmaceutically acceptable salts thereof (provided, for the aerosol or pump spray compositions, they are soluble in the spray solvent). These compounds are soluble in polar and non-polar solvents at useful concentrations. These concentrations may overlap with or be significantly less than the standard accepted dose for zolpidem. Enhanced absorption of the compounds through the oral mucosa, fast onset of action and sleep, fast onset of metabolism, and other factors contribute to the pharmaceutical efficacy of the compositions and methods for night-time dosing.

As propellants for polar and non polar sprays, propane, N-butane, iso-butane, N-pentane, iso-pentane, neo-pentane, tetrafluoroethane, heptafluoropropane, tetrafluoromethane, diethylether, dimethylether and mixtures thereof may be used. N-butane, iso-butane, HFA-134, and HFA-227 as single gases, are the preferred propellants. The propellant may be synthetically produced to minimize the presence of contaminants which may be harmful to the active compounds. Such contaminants may include oxidizing agents, reducing agents, Lewis acids or bases. The concentration of each of these should be less than 0.1%.

Optional flavoring agents include, for example, synthetic or natural mint, peppermint, spearmint, wintergreen, citrus oil, fruit flavors, sweeteners (acesulfame, aspartame, neotame, saccharin, sucralose, sugars, etc.), and combinations thereof.

The compositions may further include a taste masking agent that can hide or minimize an undesirable flavor such as a bitter or sour flavor. A representative taste mask is a combination of vanillin, ethyl vanillin, maltol, iso-amyl acetate, ethyl oxyhydrate, anisic aldehyde, and propylene glycol (commercially available as “PFC 9885 Bitter Mask” from Pharmaceutical Flavor Clinic of Camden, N.J.).

The active substances include sedatives. Suitable sedatives for use in the oral sprays of the invention include, but are not limited to, dexmedetomidine, eszopiclone, indiplon, zolpidem, and zaleplon. When zolpidem or a pharmaceutically acceptable salt thereof is the active compound the oral spray contains from about 0.01 to 20 weight/weight (w/w) percent zolpidem, about 0.1 to 15 w/w percent zolpidem, or about 0.5 to 5 w/w percent zolpidem.

When the active compound is acidic, salts may be prepared from pharmaceutically acceptable non-toxic bases. Salts derived from inorganic bases include, for example, aluminum, ammonium, calcium, copper, iron, lithium, magnesium, manganese, potassium, sodium, zinc, etc. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines and basic ion-exchange resins such as arginine, betaine, caffeine, choline, N,N dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methyl-glucosamine, morpholine, piperazine, piperidine, polyamine resins, procaine, purine, theobromine, triethylamine, trimethylamine, and tripropylamine, etc.

When the active compound is basic, salts may be prepared from pharmaceutically acceptable non-toxic acids. Such acids include acetic, benzenesulfonic, benzoic, camphorsulfonic, citric, ethane-sulfonic, fumaric, gluconic, glutamic, hydrobromic, hydrochloric, isethionic, lactic, maleic, mandelic, methanesulfonic, mucic, nitric, pamoic, pantothenic, phosphoric, succinic, sulfuric, tartaric, and p-toluenesulfonic, etc. Particularly preferred are citric, hydrobromic, hydrochloric, maleic, phosphoric, sulfuric, and tartaric acids.

In the discussion of methods of treatment herein, reference to the sedative or active compound is meant to also include the pharmaceutically acceptable salts thereof. While certain doses and formulations are set forth herein, the actual amounts to be administered to the mammal or man in need of same may be determined by the requirements of the patient, the treating physician, and/or the Food and Drug Administration.

The following examples are intended to be illustrative and not limiting. All values unless otherwise specified are in weight percent.

### Example 1

### Zolpidem Formulations

| | |
|---|---|
| Component | Percent (w/w) |

Representative propellant-free zolpidem
formulations containing a polar
solvent have the following formulas:

| | | |
|---|---|---|
| A. | Zolpidem tartrate | 4.50 |
| | Purified water | 57.44 |
| | Propylene glycol | 20.00 |
| | Citric acid anhydrous | 17.50 |
| | Flavor | 0.50 |
| | Benzoic acid | 0.05 |
| | Neotame | 0.01 |
| B. | Zolpidem tartrate | 4.66 |
| | Purified water | 48.13 |
| | Propylene glycol | 35.00 |
| | Citric acid monohydrate | 9.57 |
| | Dilute hydrochloric acid | 2.33 |
| | Flavor | 0.25 |
| | Benzoic acid | 0.05 |
| | Neotame | 0.01 |
| C. | Zolpidem tartrate | 4.80 |
| | Purified water | 54.33 |
| | Propylene glycol | 36.06 |
| | Dilute hydrochloric acid | 4.61 |
| | Flavor | 0.10 |
| | Benzoic acid | 0.05 |
| | Neotame | 0.05 |

### Example 2

### Clinical Study 1

A controlled, crossover, open-label, dose-ranging, multiple-treatment pharmacokinetic trial was conducted using a spray formulation of zolpidem. The study 1 included ten healthy fasting male volunteers aged 18 to 40 years.

Each subject received one 2.5 mg, 5 mg, and 10 mg dose of a spray formulation of zolpidem at different dosing visits. Each subject also separately received a 10 mg zolpidem tartrate (Ambien®) tablet at different dosing visits. A total of 19 blood draws per dosing visit were performed 1) at 10 minutes prior to dosing; 2) immediately following dosing; and 3) at 3, 6, 9, 12, 15, 20, 30, 45, 60, 90, 120, 180, 240, 360, 480, 600, and 720 minutes post-dosing.

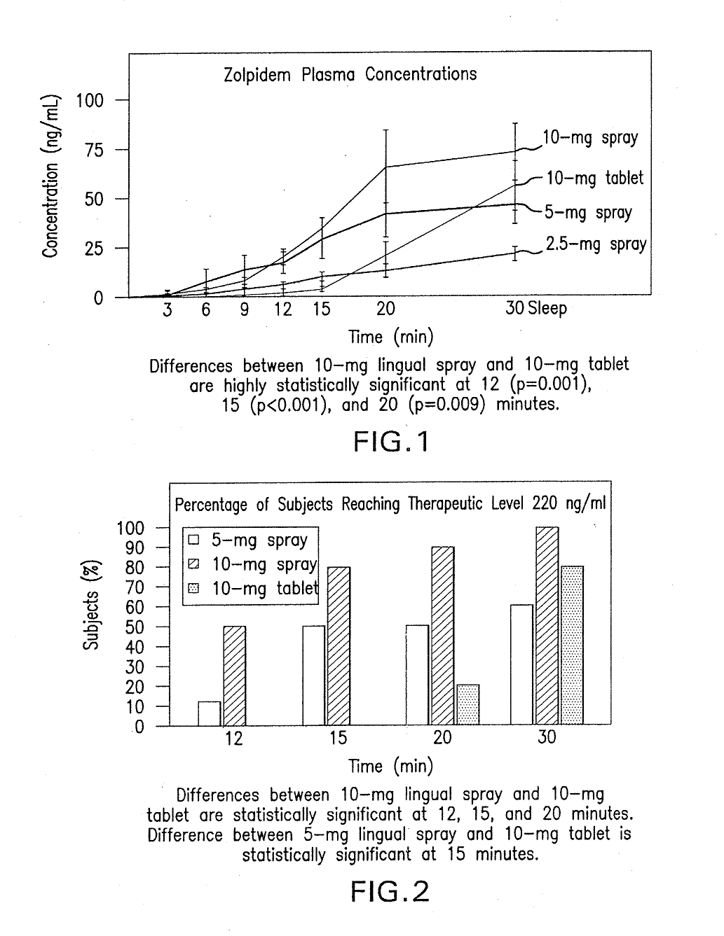

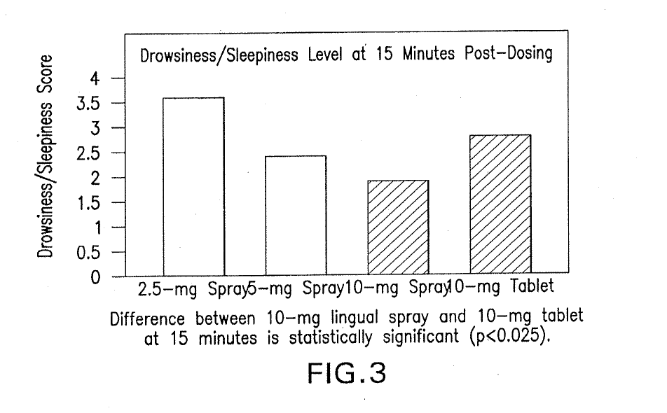

The results of study 1 are illustrated in FIGS. 1-3. Specifically, FIG. 1 displays means and standard errors of the drug concentration levels during the first 30 minutes post-dosing. The 30-minute interval is considered particularly important because it represents Ambien's® time to onset of therapeutic action as measured by sleep latency. Even without dose-adjustment, at 15 minutes post-dosing mean concentration levels were approximately 3, 8, and 9 times greater for 2.5 mg, 5 mg, and 10 mg oral sprays, respectively, compared with the oral tablet. At 12, 15, and 20 minutes post-dosing, the differences between the 10 mg spray and the oral tablet were statistically significant. At 12 and 15 minutes post-dosing, the 5 mg oral spray produced statistically significantly greater concentration levels than the 10 mg oral tablet.

Significantly, oral spray administration provides faster appearance of zolpidem in the bloodstream compared to the tablet.

### Example 3

### Clinical Studies 2 and 3

The first, single-center study using 45 healthy male and female volunteers was a randomized, 4-way crossover, open-label, dose-ranging study (Study 2). This study compared 5 mg and 10 mg doses of zolpidem oral spray to the same doses of AMBIEN® tablets. The second, single-center study using 24 elderly healthy male and female volunteers was a randomized, 2-way crossover, open-label, pharmacokinetic (PK)/pharmacodynamic (PD) study of the 5 mg zolpidem oral spray and 5 mg AMBIEN® tablet (Study 3). The study zolpidem spray formulation was as follows:

| Component | Percent (w/w) |
|---|---|
| Zolpidem tartrate, EP | 4.66 |
| Citric acid monohydrate, USP | 9.57 |
| NEOTAME ® | 0.01 |
| Diluted hydrochloric acid, NF | 2.33 |
| Propylene glycol, USP | 35.00 |
| Benzoic acid, USP/EP | 0.05 |
| W.S. artificial cherry flavor | 0.25 |
| Purified water, USP | 48.13 |

Both pharmacokinetic/pharmacodynamic studies were designed to evaluate overall comparability of the pharmacokinetic profile of the zolpidem oral spray and AMBIEN® tablets as determined by Cmax and AUCs. The studies' objectives also included comparative evaluation of metrics of the speed of drug absorption and pharmacodynamic properties of the zolpidem oral spray as well as evaluation of its safety and tolerability profile.

Data from both studies indicate overall comparability of pharmacokinetic profile of zolpidem oral spray when compared to the AMBIEN® tablet. This assessment is based on the evaluation of the maximum concentration level, Cmax and areas under the drug concentration curves, AUCs to the last measurable observation and extrapolated to the infinity.

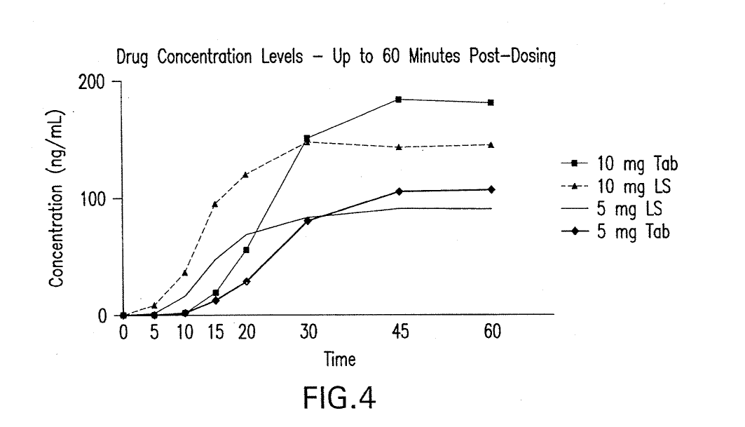

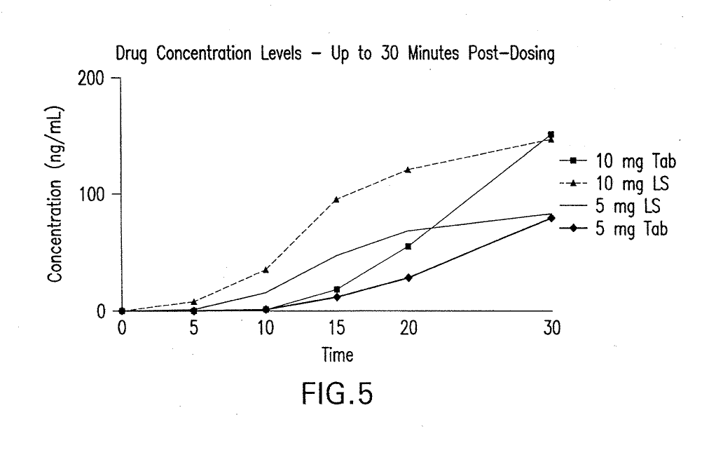

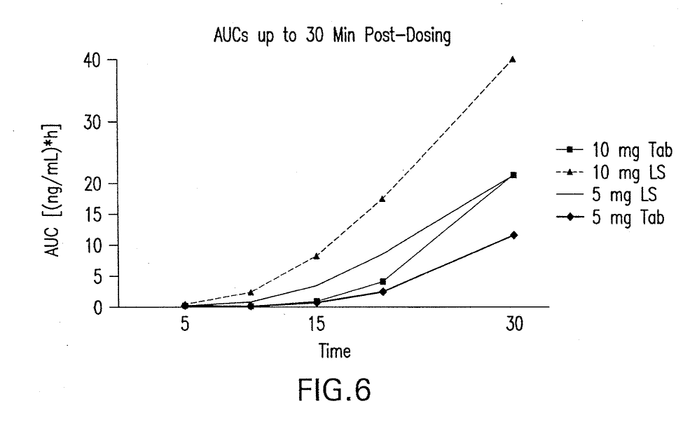

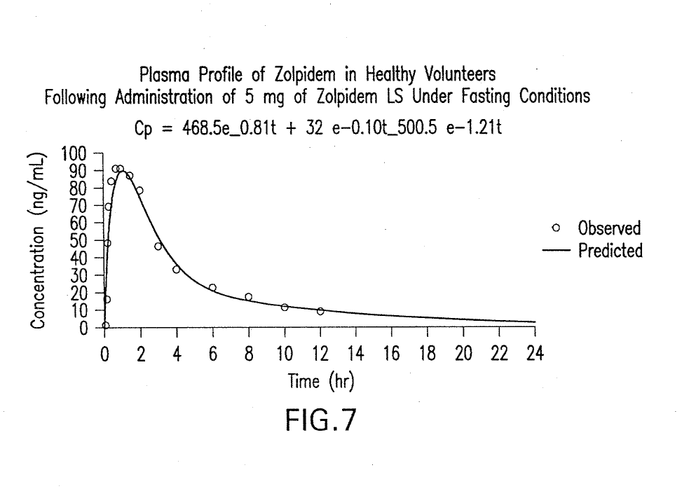

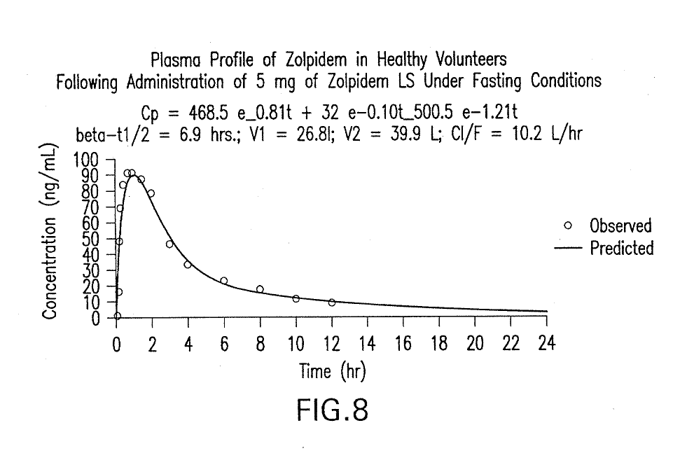

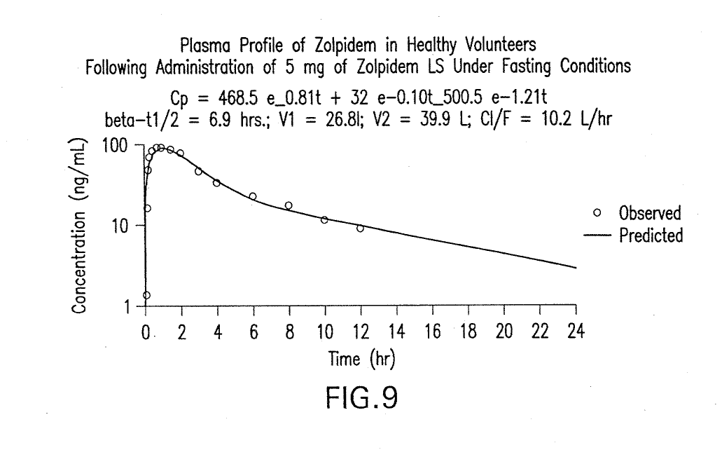

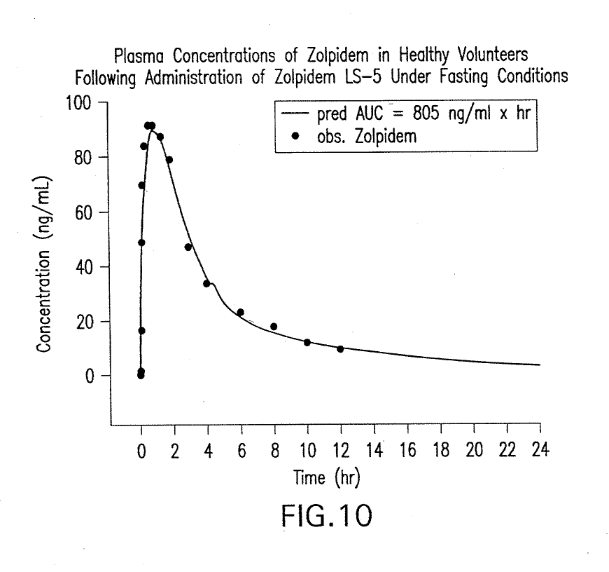

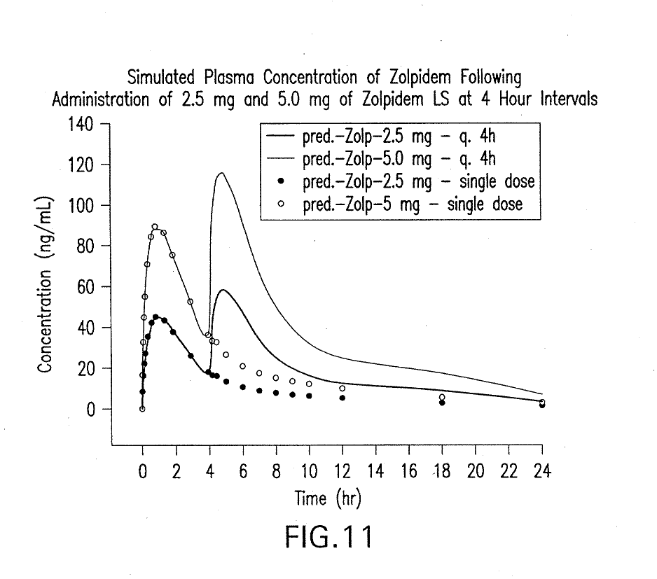

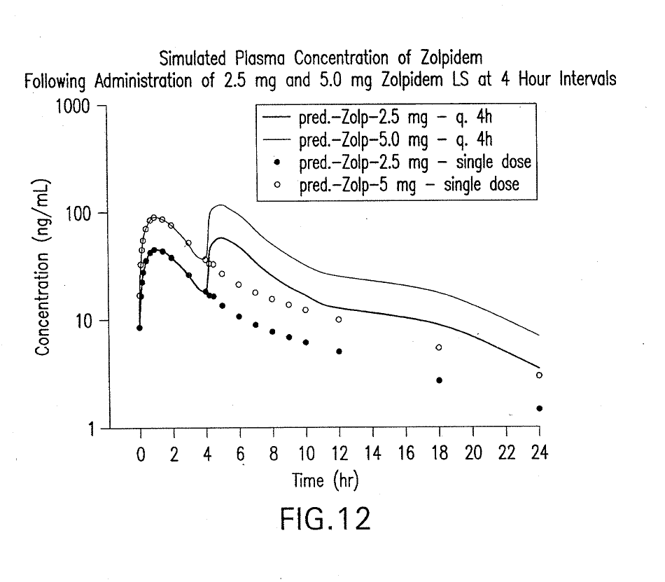

In a 4-way crossover study in 45 healthy volunteers, 64% of patients receiving 5 mg zolpidem oral spray and 78% of subjects receiving 10 mg zolpidem oral spray, reached therapeutic drug levels (>=20 ng/ML) by 15 minutes post-dosing. Results for zolpidem oral spray were statistically significantly higher when compared to 5 mg and 10 mg oral tablets with only 18% and 24% of the subjects respectively reaching therapeutic drug levels for the same 15 minute post-dosing period. Plasma zolpidem concentrations were determined by LC/MS/MS separation with consecutive detection. The results of the 4-way crossover study are shown in Tables I and II below and FIGS. 4-6.

In a 2-way crossover study in 24 geriatric volunteers (subjects older than 65 years), results were also statistically significantly higher in 5 mg zolpidem oral spray group when compared to the 5 mg oral tablet with 79% of subjects reaching therapeutic drug levels by 15 minutes post-dosing versus 29% achieving therapeutic results for the same timeframe with oral tablets. The results of the 2-way crossover study are shown in Tables III and IV below.

Evaluation of the primary pharmacodynamic endpoint, defined as the change in the Digit Symbol Substitution Test (DSST) score from pre-dosing baseline to the 13 minutes post-dosing, in both studies also revealed statistically significant superiority of the oral spray when compared to the oral tablets. Notably, 5 mg zolpidem oral spray demonstrated faster initial absorption and stronger initial pharmacodynamic effects when compared to 10 mg AMBIEN® tablets. Importantly, observed differences in the pharmacokinetic and pharmacodynamic metrics of drug absorption were not associated with increase in the overall exposure to the study drug: maximum concentration level (Cmax) and areas-under-the-curve (AUCs) were comparable between zolpidem oral spray and AMBIEN® tablets.

The oral spray groups demonstrated consistently faster drug absorption than the tablet groups as evidenced by higher concentration levels and AUCs at early post-dosing time points. For example, AUCs achieved by 15 minutes post dosing were approximately 9 times higher for the 10 mg oral spray and approximately 5 times higher for the 5 mg oral spray when compared to the same doses of AMBIEN® tablets. The primary metric of the speed of drug absorption (percentage of subjects reaching therapeutic drug levels of at least 20 ng/ml by 15 minutes post-dosing) revealed statistically significant superiority of the oral spray groups (p<0.001) when compared to the same doses of oral tablets. Notably, in the first study 64% of subjects achieved this drug level after receiving 5 mg oral spray vs. 24% of subjects dosed with 10 mg AMBIEN® tablet. This treatment difference was also highly significant (p=0.0005). Thus, the oral spray shortens the time to onset of therapeutic action as compared to a tablet.

In both studies, researchers administered the Digit Symbol Substitution Test, DSST (twice before dosing and at 13 and 23 minutes post-dosing) and 12-item Visual Analog Scale (twice before dosing and at 12 and 22 minutes post-dosing) to all participants. The DSST is a complex test, and a reduction in DSST score is considered an indicator of sleepiness and sedation. Change in the DSST from pre-dosing baseline to 13 minutes post-dosing was pre-specified as a primary pharmacodynamic endpoint in both studies. Statistically significant treatment differences were observed for this endpoint. Importantly, in the first study, 5 mg oral spray was statistically significantly superior when compared to the 10 mg AMBIEN® tablet.

Importantly, from the stand point of safety, the mean maximum plasma concentration (Cmax) and bioavailability, as measured by the area under the curve, achieved during the entire 12-hour observation period for the 10 mg oral spray did not exceed that of the oral tablet.

FIGS. 4-6 are graphs depicting plasma drug concentration levels of subjects at various time points during Study 2.

There was no evidence of any safety or tolerability issues. No adverse events were reported after administration of the oral spray doses. None of the subjects discontinued the study.

TABLE I
Study 2 BE Results

| | 5 mg | 5 mg | 10 mg | 10 mg | Ratio | 90% Conf |
|---|---|---|---|---|---|---|
| | AMBIEN | Zolpidem | AMBIEN | Zolpidem | LS/Tablet | Intervals |
| | Tablet | LS | Tablet | LS | (1) 5 mg | (1) 5 mg |
| Parameter | N = 44 | N = 44 | N = 44 | N = 44 | (2) 10 mg | (2) 10 mg |
| Cmax (ng/mL) | 114.1 | 101.1 | 206.8 | 193.0 | (1) 0.889 | (0.788-1.003) |
| LS Mean | | | | | (2) 0.933 | (0.854-1.020) |
| AUC(0-T) | 398.0 | 351.4 | 755.2 | 707.0 | (1) 0.883 | (0.789-0.991) |
| [h * (ng/mL)] | | | | | (2) 0.936 | (0.861-1.016) |
| LS Mean | | | | | | |
| AUC(0-∞) | 428.7 | 379.3 | 822.9 | 769.3 | (1) 0.885 | (0.789-0.988) |
| [h * (ng/mL)] | | | | | (2) 0.935 | (0.863-1.016) |
| LS Mean | | | | | | |

AUC(0-T) calculated by the linear trapezoidal method
AUC(0-∞) AUC(0-T) + (0.693/Kₑ)

TABLE II
Study 2 Primary PK and PD Endpoints

| | 5 mg | 5 mg | 10 mg | 10 mg | |
|---|---|---|---|---|---|
| | AMBIEN | Zolpidem | AMBIEN | Zolpidem | |
| | Tablet | LS | Tablet | LS | |
| Parameter | N = 44 | N = 44 | N = 44 | N = 44 | P-Value (Test) |
| Percentage of | 18.2% | 63.6% | 24.4% | 77.8% | P < 0.001 for all |
| Subjects Reaching | | | | | comparisons (5 mg |
| Ther Level (>=20 ng/mL) | | | | | and 10 mg LS vs 5 mg |
| by 15 Min | | | | | and 10 mg Tab) |
| | | | | | (McNemar's test) |
| Change in DSST | −3.1 ± 7.6 | −7.7 ± 8.5 | −3.3 ± 8.5 | −13.6 ± 13 | P < 0.05 For all |
| score from pre- | −1.5 | −6.5 | −1.5 | −11.5 | comparisons (5 mg |
| dosing baseline to | | | | | and 10 mg LS vs 5 mg |
| 13 Min | | | | | and 10 mg Tab): |
| Mean ± SD | | | | | (Wilcoxon Signed |
| Median | | | | | Rank, Rank |
| | | | | | ANOVA |

TABLE III
Study 3 Major PK Parameters

| | 5 mg AMBIEN Tablet | 5 mg Zolpidem LS |
|---|---|---|
| Parameter/Statistic | N = 24 | N = 24 |
| Cmax (ng/mL) | | |
| Mean ± SD | 133.7 ± 51.8 | 127.8 ± 38.4 |
| Median | 125.9 | 125.4 |
| Range | 53-268 | 52-189 |
| AUC(0-T) [h * (ng/mL)] | | |
| Mean ± SD | 457.5 ± 180.3 | 432.8 ± 180.8 |
| Median | 425.3 | 408.3 |
| Range | 187-975 | 159-913 |
| AUC(0-∞) [h * (ng/mL)] | | |
| Mean ± SD | 493.0 ± 213.2 | 465.3 ± 212.1 |
| Median | 447.4 | 423.4 |
| Range | 192-1112 | 161-1042 |

AUC(0-T) calculated by the linear trapezoidal method
AUC(0-∞) AUC(0-T) + (0.693/Kₑ)

TABLE IV
Study 3 PK and PD Endpoints

| | 5 mg | 5 mg | |
|---|---|---|---|
| | AMBIEN | Zolpidem | |
| | Tablet | LS | |
| Parameter | N = 24 | N = 24 | P-Value (Test) |
| Percentage of Subjects | 29.2% | 79.2% | P = 0.0005 |
| Reaching Ther Level | | | (McNemar's test) |
| (>=20 ng/mL) by 15 Min | | | |
| Change in DSST score | | | P = 0.0352 |
| from pre-dosing baseline | | | (Wilcoxon Signed |
| to 13 Min | | | Rank) P = 0.116 |
| Mean ± SD | 0.5 ± 7.8 | −5.4 ± 9.3 | (ANOVA)P = 0.0332 |
| Median | 1.5 | −3.3 | (Rank ANOVA) |

### Example 4

Patients suffering from insomnia would be administered a night time dose of zolpidem tartrate according to one or more of the formulations in the Studies above. The formulations can contain zolpidem in a dose of, for example, about 2.5 mg, in an oral spray composition having a unit dose volume of about 50 mcL.

At about 2:00 a.m., the patient's insomnia may be such that, although the patient would retire to bed by about 11:00 p.m. and sleep with little or no difficulty, he or she would still reawaken in the middle of the night, for example, 2:00 a.m., and be unable to fall asleep once again. Alternatively, the patient would retire to bed at about 2:00 a.m. without having tried to sleep earlier, but may believe that an anti-insomnia medication would be necessary to fall asleep or achieve any meaningful degree of restful sleep before awakening again in about 4 to 5 hours. In either event, a night time dose of the above referenced zolpidem formulation would be administered by oral spray, even though the patient must arise and resume wakeful activities at say 6:00 a.m., approximately 4 to 5 hours after receiving the anti-insomnia, middle of the night, therapeutic dose by oral spray.

Such wakeful activities would include, for example, working or exercising. These wakeful activities would be conducted without any undue after-effects from the anti-insomnia medication and composition delivered by oral spray. The blood plasma levels upon awakening at 6:00 a.m. would be below therapeutic levels, i.e., below about 20 ng/ml.

1-23. (canceled)

24. A pharmaceutical anti-insomnia composition comprising:
zolpidem or a pharmaceutically acceptable salt thereof; and
a pharmaceutically acceptable solvent;
wherein said composition is contained in a unit dose spray pump container;
and wherein a single actuation of said container delivers a unit dose volume about 50 to about 400 mcL of said composition containing a dose of about 0.5 to 5 mg zolpidem or a pharmaceutically acceptable salt thereof.

25. The composition of claim 24, wherein the unit dose volume is about 50 to about 200 mcL.

26. The composition of claim 24, wherein the dose of zolpidem is about 2 to 3 mg.

27. The composition of claim 24, wherein upon delivery of the unit dose to a human patient by oral spray a therapeutic zolpidem level is achieved within less than 30 minutes post dosing.

28. The composition of claim 24, wherein upon delivery of the unit dose to a human patient by oral spray a therapeutic zolpidem level is achieved within 23 minutes post dosing.

29. The composition of claim 24, wherein upon delivery of the unit dose to a human patient by oral spray a therapeutic zolpidem level is achieved within 22 minutes post dosing.

30. The composition of claim 24, wherein upon delivery of the unit dose to a human patient by oral spray a therapeutic zolpidem level is achieved within 13 minutes post dosing.

31. The composition of claim 24, wherein upon delivery of the unit dose to a human patient by oral spray a therapeutic zolpidem level is achieved within 12 minutes post dosing.

32. The composition of claim 24, wherein upon delivery of the unit dose to a human patient by oral spray zolpidem blood levels taper off to less than 20 ng/ml in less than five hours post dosing.

33. The composition of claim 24, wherein upon delivery of the unit dose to a human patient by oral spray zolpidem blood levels taper off to less than 20 ng/ml in less than four hours post dosing.
